(19) United States
(12) Patent Application Publication
(10) Pub. No.: US 2025/0034090 A1
(43) Pub. Date: Jan. 30, 2025

(54) NOVEL S1P1 RECEPTOR AGONIST, CRYSTALLINE SALTS, PROCESSES FOR PREPARING, AND USES RELATED THERETO

(71) Applicant: Arena Pharmaceuticals, Inc., New York, NY (US)

(72) Inventors: Anthony C. Blackburn, San Diego, CA (US); Jui-Chen LIN, Taipei City (TW)

(73) Assignee: Arena Pharmaceuticals, Inc., New York, NY (US)

(21) Appl. No.: 18/841,455

(22) PCT Filed: Mar. 7, 2023
(86) PCT No.: PCT/IB2023/052160

§ 371 Date: Aug. 26, 2024

Related U.S. Application Data
(60) Provisional application No. 63/318,329, filed on Mar. 9, 2022.
(60) Provisional application No. 63/485,708, filed on Feb. 17, 2023.

(51) Int. Cl. C07D 209/60; A61K 31/404; C07C 211/05; C07C 215/08

(52) U.S. Cl. CPC C07D 209/60; A61K 31/404; C07C 211/05; C07C 215/08

(57) ABSTRACT

The present invention relates to crystalline salts of (R)-2-(7-(4-cyclopentyl-3-(trifluoromethyl)benzyloxy)-1,2,3,4-tetrahydrocyclo-penta[b]indol-3-yl)acetic acid, and pharmaceutical compositions comprising them that are useful as SiP1 receptor modulator. The compound (R)-2-(9-chloro-7-(4-isopropoxy-3-(trifluoromethyl)benzyloxy)-2,3-dihydro-1H-pyrrolo[1,2-a]indol-1-yl)acetic acid identified as an SiP1 receptor modulator is useful in the treatment of SiP1 receptor-associated disorders, for example, diseases and disorders mediated by lymphocytes, transplant rejection, autoimmune diseases and disorders, inflammatory diseases and disorders (e.g., acute and chronic inflammatory conditions), cancer, and conditions characterized by an underlying defect in vascular integrity or that are associated with angiogenesis such as may be pathologic (e.g., as may occur in inflammation, tumor development, and atherosclerosis).

### FIELD OF THE INVENTION

The present invention relates to crystalline salts of (R)-2-(7-(4-cyclopentyl-3-(trifluoromethyl)benzyloxy)-1,2,3,4-tetrahydrocyclo-penta[b]indol-3-yl)acetic acid (Compound 1), and pharmaceutical compositions comprising them that are useful for, inter alia, in the treatment of S1P, receptor-associated disorders, for example, diseases and disorders mediated by lymphocytes, transplant rejection, autoimmune diseases and disorders, inflammatory diseases and disorders (e.g., acute and chronic inflammatory conditions), cancer, and conditions characterized by an underlying defect in the vascular integrity or that are associated with pathological angiogenesis (e.g., as may occur in inflammation, tumor development, and atherosclerosis).

### BACKGROUND OF THE INVENTION

The present invention relates to compounds that are S1P1 receptor agonists having at least immunosuppressive, anti-inflammatory and/or hemostatic activities, e.g. by virtue of modulating leukocyte trafficking, sequestering lymphocytes in secondary lymphoid tissues, and/or enhancing vascular integrity.

The present application is in part focused on addressing an unmet need for immunosuppressive agents such as may be orally available which have therapeutic efficacy for at least autoimmune diseases and disorders, inflammatory diseases and disorders (e.g., acute and chronic inflammatory conditions), transplant rejection, cancer, and/or conditions that have an underlying defect in vascular integrity or that are associated with angiogenesis such as may be pathologic (e.g., as may occur in inflammation, tumor development and atherosclerosis) with fewer side effects such as the impairment of immune responses to systemic infection.

The sphingosine-1-phosphate (S1P) receptors 1-5 constitute a family of G protein-coupled receptors with a seven-transmembrane domain. These receptors, referred to as S1P1 to S1P5 (formerly termed endothelial differentiation gene (EDG) receptor-1, -5, -3, -6 and -8, respectively; Chun et al., Pharmacological Reviews, 54:265-269, 2002), are activated via binding by sphingosine-1-phosphate, which is produced by the sphingosine kinase-catalyzed phosphorylation of sphingosine. S1P1, S1P4 and S1P5 receptors activate Gi but not Gq, whereas S1P2 and S1P3 receptors activate both Gi and Gq. The S1P3 receptor, but not the S1P1 receptor, responds to an agonist with an increase in intracellular calcium.

S1P receptor agonists having agonist activity on the S1P1 receptor have been shown to rapidly and reversibly induce lymphopenia (also referred to as peripheral lymphocyte lowering (PLL); Hale et al., Bioorg. Med. Chem. Lett., 14:3351-3355, 2004). This is attended by clinically useful immunosuppression by virtue of sequestering T- and B-cells in secondary lymphoid tissue (lymph nodes and Peyer's patches) and thus apart from sites of inflammation and organ grafts (Rosen et al., Immunol. Rev., 195:160-177, 2003; Schwab et al., Nature Immunol., 8:1295-1301, 2007). This lymphocyte sequestration, for example in lymph nodes, is thought to be a consequence of concurrent agonist-driven functional antagonism of the S1P1 receptor on T-cells (whereby the ability of S1P to mobilize T-cell egress from lymph nodes is reduced) and persistent agonism of the S1P1 receptor on lymph node endothelium (such that barrier function opposing transmigration of lymphocytes is increased) (Matloubian et al., Nature, 427:355-360, 2004; Baumruker et al., Expert Opin. Investig. Drugs, 16:283-289, 2007). It has been reported that agonism of the S1P1 receptor alone is sufficient to achieve lymphocyte sequestration (Sanna et al., J Biol Chem., 279:13839-13848, 2004) and that this occurs without impairment of immune responses to systemic infection (Brinkmann et al., Transplantation, 72:764-769, 2001; Brinkmann et al., Transplant Proc., 33:530-531, 2001).

That agonism of endothelial S1P1 receptors has a broader role in promoting vascular integrity is supported by work implicating the S1P1 receptor in capillary integrity in mouse skin and lung (Sanna et al., Nat Chem Biol., 2:434-441, 2006). Vascular integrity can be compromised by inflammatory processes, for example as may derive from sepsis, major trauma and surgery so as to lead to acute lung injury or respiratory distress syndrome (Johan Groeneveld, Vascul. Pharmacol., 39:247-256, 2003).

An exemplary S1P receptor agonist having agonist activity on the S1P1 receptor is FTY720 (fingolimod), an immunosuppressive agent currently in clinical trials (Martini et al., Expert Opin. Investig. Drugs, 16:505-518, 2007). FTY720 acts as a prodrug which is phosphorylated in vivo; the phosphorylated derivative is an agonist for S1P1, S1P3, S1P4 and S1P5 receptors (but not the S1P2 receptor) (Chiba, Pharmacology & Therapeutics, 108:308-319, 2005). FTY720 has been shown to rapidly and reversibly induce lymphopenia (also referred to as peripheral lymphocyte lowering (PLL); Hale et al., Bioorg. Med. Chem. Lett., 14:3351-3355, 2004). This is attended by clinically useful immunosuppression by virtue of sequestering T- and B-cells in secondary lymphoid tissue (lymph nodes and Peyer's patches) and thus apart from sites of inflammation and organ grafts (Rosen et al., Immunol. Rev., 195:160-177, 2003; Schwab et al., Nature Immunol., 8:1295-1301, 2007).

In clinical trials, FTY720 elicited an adverse event (i.e., transient asymptomatic bradycardia) due to its agonism of the S1P3 receptor (Budde et al., J. Am. Soc. Nephrol., 13:1073-1083, 2002; Sanna et al., J. Biol. Chem., 279:13839-13848, 2004; Ogawa et al., BBRC, 361:621-628, 2007).

FTY720 has been reported to have therapeutic efficacy in at least: a rat model for autoimmune myocarditis and a mouse model for acute viral myocarditis (Kiyabayashi et al., J. Cardiovasc. Pharmacol., 35:410-416, 2000; Miyamoto et al., J. Am. Coll. Cardiol., 37:1713-1718, 2001); mouse models for inflammatory bowel disease including colitis (Mizushima et al., Inflamm. Bowel Dis., 10:182-192, 2004; Deguchi et al., Oncology Reports, 16:699-703, 2006; Fujii et al., Am. J. Physiol. Gastrointest. Liver Physiol., 291:G267-G274, 2006; Daniel et al., J. Immunol., 178:2458-2468, 2007); a rat model for progressive mesangioproliferative glomerulonephritis (Martini et al., Am. J. Physiol. Renal Physiol., 292:F1761-F1770, 2007); a mouse model for asthma, suggested to be primarily through the S1P1 receptor on the basis of work using the the S1P1 receptor agonist SEW2871 (Idzko et al, J. Clin. Invest., 116:2935-2944, 2006); a mouse model for airway inflammation and induction of bronchial hyperresponsiveness (Sawicka et al., J. Immunol., 171; 6206-6214, 2003); a mouse model for atopic dermatitis (Kohno et al., Biol. Pharm. Bull., 27:1392-1396, 2004); a mouse model for ischemia-reperfusion injury (Kaudel et al., Transplant. Proc, 39:499-502, 2007); a mouse model for systemic lupus erythematosus (SLE) (Okazaki et al., J. Rheumatol., 29:707-716, 2002; Herzinger et al, Am. J. Clin. Dermatol., 8:329-336, 2007); rat models for rheumatoid arthritis (Matsuura et al., Int. J. Immunopharmacol., 22:323-331, 2000; Matsuura et al., Inflamm. Res., 49:404-410, 2000); a rat model for autoimmune uveitis (Kurose et al., Exp. Eye Res., 70:7-15, 2000); mouse models for type I diabetes (Fu et al, Transplantation, 73:1425-1430, 2002; Maki et al., Transplantation, 74:1684-1686, 2002; Yang et al., Clinical Immunology, 107:30-35, 2003; Maki et al., Transplantation, 79:1051-1055, 2005); mouse models for atherosclerosis (Nofer et al., Circulation, 115:501-508, 2007; Keul et al., Arterioscler. Thromb. Vasc. Biol., 27:607-613, 2007); a rat model for brain inflammatory reaction following traumatic brain injury (TBI) (Zhang et al., J. Cell. Mol. Med., 11:307-314, 2007); and mouse models for graft coronary artery disease and graft-versus-host disease (GVHD) (Hwang et al., Circulation, 100:1322-1329, 1999; Taylor et al., Blood, 110:3480-3488, 2007). In vitro results suggest that FTY720 may have therapeutic efficacy for p-amyloid-related inflammatory diseases including Alzheimer's disease (Kaneider et al., FASEB J., 18:309-311, 2004). KRP-203, an S1P receptor agonist having agonist activity on the S1P1 receptor, has been reported to have therapeutic efficacy in a rat model for autoimmune myocarditis (Ogawa et al., BBRC, 361:621-628, 2007). Using the S1P1 receptor agonist SEW2871, it has been shown that agonism of endothelial S1P1 receptors prevents proinflammatory monocyte/endothelial interactions in type I diabetic vascular endothelium (Whetzel et al., Circ. Res., 99:731-739, 2006) and protects the vasculature against TNFα-mediated monocyte/endothelial interactions (Bolick et al., Arterioscler. Thromb. Vasc. Biol., 25:976-981, 2005).

Additionally, FTY720 has been reported to have therapeutic efficacy in experimental autoimmune encephalomyelitis (EAE) in rats and mice, a model for human multiple sclerosis (Brinkmann et al., J. Biol. Chem., 277:21453-21457, 2002; Fujino et al., J. Pharmacol. Exp. Ther., 305:70-77, 2003; Webb et al., J. Neuroimmunol., 153:108-121, 2004; Rausch et al., J. Magn. Reson. Imaging, 20:16-24, 2004; Kataoka et al., Cellular & Molecular Immunology, 2:439-448, 2005; Brinkmann et al., Pharmacology & Therapeutics, 115:84-105, 2007; Baumruker et al., Expert Opin. Investig. Drugs, 16:283-289, 2007; Balatoni et al., Brain Research Bulletin, 74:307-316, 2007). Furthermore, FTY720 has been found to have therapeutic efficacy for multiple sclerosis in clinical trials. In Phase II clinical trials for relapsing-remitting multiple sclerosis, FTY720 was found to reduce the number of lesions detected by magnetic resonance imaging (MRI) and clinical disease activity in patients with multiple sclerosis (Kappos et al., N. Engl. J. Med., 355:1124-1140, 2006; Martini et al., Expert Opin. Investig. Drugs, 16:505-518, 2007; Zhang et al., Mini-Reviews in Medicinal Chemistry, 7:845-850, 2007; Brinkmann, Pharmacology & Therapeutics, 115:84-105, 2007). FTY720 is currently in Phase Ill studies of remitting-relapsing multiple sclerosis (Brinkmann, Pharmacology & Therapeutics, 115:84-105, 2007; Baumruker et al., Expert. Opin. Investig. Drugs, 16:283-289, 2007; Dev et al., Pharmacology and Therapeutics, 117:77-93, 2008).

Recently, FTY720 has been reported to have anti-viral activity. Specific data has been presented in the lymphocytic choriomeningitis virus (LCMV) mouse model, wherein the mice were infected with either the Armstrong or the clone 13 strain of LCMV (Premenko-Lanier et al., Nature, 454, 894, 2008).

FTY720 has been reported to impair migration of dendritic cells infected with Francisella tularensis to the mediastinal lymph node, thereby reducing the bacterial colonization of it. Francisella tularensis is associated with tularemia, ulceroglandular infection, respiratory infection and a typhoidal disease (E. Bar-Haim et al, PLoS Pathogens, 4(11): e1000211. doi:10.1371/journal.ppat.1000211, 2008).

It has also been recently reported that a short-term high dose of FTY720 rapidly reduced ocular infiltrates in experimental autoimmune uveoretinitis. When given in the early stages of ocular inflammation, FTY720 rapidly prevented retinal damage. It was reported to not only prevent infiltration of target organs, but also reduce existing infiltration (Raveney et al., Arch. Ophthalmol. 126(10), 1390, 2008).

It has been reported that treatment with FTY720 relieved ovariectomy-induced osteoporosis in mice by reducing the number of mature osteoclasts attached to the bone surface. The data provided evidence that S1P controled the migratory behaviour of osteoclast precursors, dynamically regulating bone mineral homeostasis (Ishii et al., Nature, advance online publication, 8 Feb. 2009, doi:10.1038/nature07713).

Agonism of the S1P1 receptor has been implicated in enhancement of survival of oligodendrocyte progenitor cells. Survival of oligodendrocyte progenitor cells is a required component of the remyelination process. Remyelination of multiple sclerosis lesions is considered to promote recovery from clinical relapses. (Miron et al., Ann. Neurol., 63:61-71, 2008; Coelho et al., J. Pharmacol. Exp. Ther., 323:626-635, 2007; Dev et al., Pharmacology and Therapeutics, 117:77-93, 2008). It also has been shown that the S1P1 receptor plays a role in platelet-derived growth factor (PDGF)-induced oligodendrocyte progenitor cell mitogenesis (Jung et al., Glia, 55:1656-1667, 2007).

Agonism of the S1P1 receptor has also been reported to mediate migration of neural stem cells toward injured areas of the central nervous system (CNS), including in a rat model of spinal cord injury (Kimura et al., Stem Cells, 25:115-124, 2007).

Agonism of the S1P1 receptor has been implicated in the inhibition of keratinocyte proliferation (Sauer et al., J. Biol. Chem., 279:38471-38479, 2004), consistent with reports that S1P inhibits keratinocyte proliferation (Kim et al., Cell Signal, 16:89-95, 2004). The hyperproliferation of keratinocytes at the entrance to the hair follicle, which can then become blocked, and an associated inflammation are significant pathogenetic factors of acne (Koreck et al., Dermatology, 206:96-105, 2003; Webster, Cutis, 76:4-7, 2005).

FTY720 has been reported to have therapeutic efficacy in inhibiting pathologic angiogenesis, such as that as may occur in tumor development. Inhibition of angiogenesis by FTY720 is thought to involve agonism of the S1P1 receptor (Oo et al., J. Biol. Chem., 282; 9082-9089, 2007; Schmid et al., J. Cell Biochem., 101:259-270, 2007). FTY720 has been reported to have therapeutic efficacy for inhibiting primary and metastatic tumor growth in a mouse model of melanoma (LaMontagne et al., Cancer Res., 66:221-231, 2006). FTY720 has been reported to have therapeutic efficacy in a mouse model for metastatic hepatocellular carcinoma (Lee et al., Clin. Cancer Res., 11:84588466, 2005).

It has been reported that oral administration of FTY720 to mice potently blocked VEGF-induced vascular permeability, an important process associated with angiogenesis, inflammation, and pathological conditions such as sepsis, hypoxia, and solid tumor growth (T Sanchez et al, J. Biol. Chem., 278(47), 47281-47290, 2003).

Cyclosporin A and FK506 (calcineurin inhibitors) are drugs used to prevent rejection of transplanted organs. Although they are effective in delaying or suppressing transplant rejection, classical immunosuppressants such as cyclosporin A and FK506 are known to cause several undesirable side effects including nephrotoxicity, neurotoxicity, p-cell toxicity and gastrointestinal discomfort. There is an unmet need in organ transplantation for an immunosuppressant without these side effects which is effective as a monotherapy or in combination with a classical immunosuppressant for inhibiting migration of, e.g., alloantigen-reactive T-cells to the grafted tissue, thereby prolonging graft survival.

FTY720 has been shown to have therapeutic efficacy in transplant rejection both as a monotherapy and in synergistic combination with a classical immunosuppressant, including cyclosporin A, FK506 and RAD (an mTOR inhibitor). It has been shown that, unlike the classical immunosuppressants cyclosporin A, FK506 and RAD, FTY720 has efficacy for prolonging graft survival without inducing general immunosuppression, and this difference in drug action is believed to be relevant to the synergism observed for the combination (Brinkmann et al., Transplant Proc., 33:530-531, 2001; Brinkmann et al., Transplantation, 72:764-769, 2001).

Agonism of the S1P1 receptor has been reported to have therapeutic efficacy for prolonging allograft survival in mouse and rat skin allograft models (Lima et al., Transplant Proc., 36:1015-1017, 2004; Yan et al., Bioorg. & Med. Chem. Lett., 16:3679-3683, 2006). FTY720 has been reported to have therapeutic efficacy for prolonging allograft survival in a rat cardiac allograft model (Suzuki et al., Transpl. Immunol., 4:252-255, 1996). FTY720 has been reported to act synergistically with cyclosporin A to prolong rat skin allograft survival (Yanagawa et al., J. Immunol., 160:5493-5499, 1998), to act synergistically with cyclosporin A and with FK506 to prolong rat cardiac allograft survival, and to act synergistically with cyclosporin A to prolong canine renal allograft survival and monkey renal allograft survival (Chiba et al., Cell Mol. Biol., 3:11-19, 2006). KRP-203, an S1P receptor agonist has been reported to have therapeutic efficacy for prolonging allograft survival in a rat skin allograft model and both as monotherapy and in synergistic combination with cyclosporin A in a rat cardiac allograft model (Shimizu et al., Circulation, 111:222-229, 2005). KRP-203 also has been reported to have therapeutic efficacy in combination with mycophenolate mofetil (MMF; a prodrug for which the active metabolite is mycophenolic acid, an inhibitor of purine biosynthesis) for prolonging allograft survival both in a rat renal allograft model and in a rat cardiac allograft model (Suzuki et al., J. Heart Lung Transplant, 25:302-209, 2006; Fujishiro et al., J. Heart Lung Transplant, 25:825-833, 2006). It has been reported that an agonist of the S1P1 receptor, AUY954, in combination with a subtherapeutic dose of RAD001 (Certican/Everolimus, an mTOR inhibitor) can prolong rat cardiac allograft survival (Pan et al., Chemistry & Biology, 13:1227-1234, 2006). In a rat small bowel allograft model, FTY720 has been reported to act synergistically with cyclosporin A to prolong small bowel allograft survival (Sakagawa et al., Transpl. Immunol., 13:161-168, 2004). FTY720 has been reported to have therapeutic efficacy in a mouse islet graft model (Fu et al., Transplantation, 73:1425-1430, 2002; Liu et al., Microsurgery, 27:300-304; 2007) and in a study using human islet cells to evidence no detrimental effects on human islet function (Truong et al., American Journal of Transplantation, 7:2031-2038, 2007).

FTY720 has been reported to reduce the nociceptive behavior in the spared nerve injury model for neuropathic pain which does not depend on prostaglandin synthesis (O. Costu et al, Journal of Cellular and Molecular Medicine 12(3), 995-1004, 2008).

FTY720 has been reported to impair initiation of murine contact hypersensitivity (CHS). Adoptive transfer of immunized lymph node cells from mice treated with FTY720 during the sensitization phase was virtually incapable of inducing CHS response in recipients (D. Nakashima et al., J. Investigative Dermatology (128(12), 2833-2841, 2008).

It has been reported that prophylactic oral administration of FTY720 (1 mg/kg, three times a week), completely prevented the development of experimental autoimmune myasthenia gravis (EAMG) in C57BL/6 mice (T. Kohono et al, Biological & Pharmaceutical Bulletin, 28(4), 736-739, 2005).

In one embodiment, the present invention encompasses compounds which are agonists of the S1P1 receptor having selectivity over the S1P3 receptor. The S1P3 receptor, and not the S1P1 receptor, has been directly implicated in bradycardia (Sanna et al., J. Biol. Chem., 279:13839-13848, 2004). An S1P1 receptor agonist selective over at least the S1P3 receptor has advantages over current therapies by virtue of an enhanced therapeutic window, allowing better tolerability with higher dosing and thus improving efficacy as therapy. The present invention encompasses compounds which are agonists of the S1P1 receptor and which exhibit no or substantially no activity for bradycardia.

S1P1 receptor agonists are useful to treat or prevent conditions where suppression of the immune system or agonism of the S1P1 receptor is in order, such as diseases and disorders mediated by lymphocytes, transplant rejection, autoimmune diseases and disorders, inflammatory diseases and disorders, and conditions that have an underlying defect in vascular integrity or that relate to angiogenesis such as may be pathologic.

In one embodiment, the present invention encompasses compounds which are agonists of the S1P1 receptor having good overall physical properties and biological activities and having an effectiveness that is substantially at least that of prior compounds with activity at the S1P1 receptor.

Citation of any reference throughout this application is not to be construed as an admission that such reference is prior art to the present application.

### SUMMARY OF THE INVENTION

A priori, it is difficult to predict with confidence which salts of a particular compound will be solid, stable, and readily isolable. Salts of chiral amines with optically active acids are no exception. It is only through diligent experimentation that a stable, solid-state salt may be discovered; once that salt is in-hand however, the artisan of ordinary skill is equipped to resolve its diastereoisomers. In the course of preparing the salts of the present invention, many salt-forming agents commonly used in the pharmaceutical industry (see e.g. Berge, et al., Journal of Pharmaceutical Sciences, 66:1-19 (1977)) were investigated.

One aspect of the present invention pertains to certain amine-based crystalline salts of (R)-2-(7-(4-cyclopentyl-3-(trifluoromethyl)benzyloxy)-1,2,3,4-tetrahydrocyclo-penta[b]indol-3-yl)acetic acid (Compound 1) and pharmaceutically acceptable solvates and hydrates thereof.

One aspect of the present invention pertains to certain amine-based crystalline salts of (R)-2-(7-(4-cyclopentyl-3-(trifluoromethyl)benzyloxy)-1,2,3,4-tetrahydrocyclo-penta[b]indol-3-yl)acetic acid (Compound 1).

One aspect of the present invention pertains to crystalline salts selected from: (R)-2-(7-(4-cyclopentyl-3-(trifluoromethyl)benzyloxy)-1,2,3,4-tetrahydrocyclo-penta[b]indol-3-yl)acetic acid diethanolamine salt; (R)-2-(7-(4-cyclopentyl-3-(trifluoromethyl)benzyloxy)-1,2,3,4-tetrahydrocyclo-penta[b]indol-3-yl)acetic acid diethylamine salt; and pharmaceutically acceptable solvates and hydrates thereof.

One aspect of the present invention is directed to (R)-2-(7-(4-cyclopentyl-3-(trifluoromethyl)benzyloxy)-1,2,3,4-tetrahydrocyclo-penta[b]indol-3-yl)acetic acid diethanolamine salt.

One aspect of the present invention is directed to (R)-2-(7-(4-cyclopentyl-3-(trifluoromethyl)benzyloxy)-1,2,3,4-tetrahydrocyclo-penta[b]indol-3-yl)acetic acid diethylamine salt.

One aspect of the present invention is directed to compositions comprising a crystalline salt, each as described herein.

One aspect of the present invention is directed to pharmaceutical compositions comprising a crystalline salt, each as described herein, and a pharmaceutically acceptable carrier.

One aspect of the present invention is directed to methods for treating an S1P1 receptor-associated disorder in an individual comprising administering to the individual in need thereof a therapeutically effective amount of a crystalline salt, or a pharmaceutical composition, each as described herein.

One aspect of the present invention is directed to methods for treating a disease or disorder mediated by lymphocytes in an individual comprising administering to the individual in need thereof a therapeutically effective amount of a crystalline salt, or a pharmaceutical composition, each as described herein.

One aspect of the present invention is directed to methods for treating an autoimmune disease or disorder in an individual comprising administering to the individual in need thereof a therapeutically effective amount of a crystalline salt, or a pharmaceutical composition, each as described herein.

One aspect of the present invention is directed to methods for treating an inflammatory disease or disorder in an individual comprising administering to the individual in need thereof a therapeutically effective amount of a crystalline salt, or a pharmaceutical composition, each as described herein.

One aspect of the present invention is directed to methods for treating a microbial infection or disease in an individual comprising administering to the individual in need thereof a therapeutically effective amount of a crystalline salt, or a pharmaceutical composition, each as described herein.

One aspect of the present invention is directed to methods for treating a viral infection or disease in an individual comprising administering to the individual in need thereof a therapeutically effective amount of a crystalline salt, or a pharmaceutical composition, each as described herein.

One aspect of the present invention is directed to methods for treating cancer in an individual comprising administering to the individual in need thereof a therapeutically effective amount of a crystalline salt, or a pharmaceutical composition, each as described herein.

One aspect of the present invention is directed to methods for treating a disorder in an individual comprising administering to said individual in need thereof a therapeutically effective amount of a crystalline salt, or a pharmaceutical composition, each as described herein, wherein said disorder is selected from the group consisting of psoriasis, rheumatoid arthritis, Crohn's disease, transplant rejection, multiple sclerosis, systemic lupus erythematosus, ulcerative colitis, type I diabetes, acne, myocardial ischemia-reperfusion injury, hypertensive nephropathy, glomerulosclerosis, gastritis, polymyositis, thyroiditis, vitiligo, hepatitis, and biliary cirrhosis.

One aspect of the present invention pertains to the use of a crystalline salt, each as described herein, in the manufacture of a medicament for the treatment of an S1P1 receptor-associated disorder.

One aspect of the present invention pertains to the use of a crystalline salt, each as described herein, in the manufacture of a medicament for the treatment of a disease or disorder mediated by lymphocytes.

One aspect of the present invention pertains to the use of a crystalline salt, each as described herein, in the manufacture of a medicament for the treatment of an autoimmune disease or disorder.

One aspect of the present invention pertains to the use of a crystalline salt, each as described herein, in the manufacture of a medicament for the treatment of an inflammatory disease or disorder.

One aspect of the present invention pertains to the use of a salt, each as described herein, in the manufacture of a medicament for the treatment of a microbial infection o crystalline r disease.

One aspect of the present invention pertains to the use of a crystalline salt, each as described herein, in the manufacture of a medicament for the treatment of a viral infection or disease.

One aspect of the present invention pertains to the use of a crystalline salt, each as described herein, in the manufacture of a medicament for the treatment of cancer.

One aspect of the present invention pertains to the use of a crystalline salt, each as described herein, in the manufacture of a medicament for the treatment of an S1P1 receptor-associated disorder selected from the group consisting of psoriasis, rheumatoid arthritis, Crohn's disease, transplant rejection, multiple sclerosis, systemic lupus erythematosus, ulcerative colitis, type I diabetes, acne, myocardial ischemia-reperfusion injury, hypertensive nephropathy, glomerulosclerosis, gastritis, polymyositis, thyroiditis, vitiligo, hepatitis, and biliary cirrhosis.

One aspect of the present invention pertains to a crystalline salt, or pharmaceutical composition, each as described herein, for use in a method for the treatment of the human or animal body by therapy.

One aspect of the present invention pertains to a crystalline salt, or pharmaceutical composition, each as described herein, for use in a method for the treatment of an S1P1 receptor-associated disorder.

One aspect of the present invention pertains to a crystalline salt, or pharmaceutical composition, each as described herein, for use in a method for the treatment of a disease or disorder mediated by lymphocytes.

One aspect of the present invention pertains to a crystalline salt, or pharmaceutical composition, each as described herein, for use in a method for the treatment of an autoimmune disease or disorder.

One aspect of the present invention pertains to a crystalline salt, or pharmaceutical composition, each as described herein, for use in a method for the treatment of an inflammatory disease or disorder.

One aspect of the present invention pertains to a crystalline salt, or pharmaceutical composition, each as described herein, for use in a method for the treatment of a microbial infection or disease.

One aspect of the present invention pertains to a crystalline salt, or pharmaceutical composition, each as described herein, for use in a method for the treatment of a viral infection or disease.

One aspect of the present invention pertains to a crystalline salt, a crystalline form, or pharmaceutical composition, each as described herein, for use in a method for the treatment of cancer.

One aspect of the present invention pertains to a crystalline salt, or pharmaceutical composition, each as described herein, for use in a method for the treatment of an S1P1 receptor-associated disorder selected from the group consisting of psoriasis, rheumatoid arthritis, Crohn's disease, transplant rejection, multiple sclerosis, systemic lupus erythematosus, ulcerative colitis, type I diabetes, acne, myocardial ischemia-reperfusion injury, hypertensive nephropathy, glomerulosclerosis, gastritis, polymyositis, thyroiditis, vitiligo, hepatitis, and biliary cirrhosis.

One aspect of the present invention pertains to processes for preparing compositions comprising admixing a crystalline salt, each as described herein, and a pharmaceutically acceptable carrier.

One aspect of the present invention pertains to a crystalline salt, or pharmaceutical composition, each as described herein, in the manufacture of a medicament for the treatment of an S1P1 receptor-associated disorder.

One aspect of the present invention pertains to a crystalline salt, or pharmaceutical composition, each as described herein, in the manufacture of a medicament for the treatment of a disease or disorder mediated by lymphocytes.

One aspect of the present invention pertains to a crystalline salt, or pharmaceutical composition, each as described herein, in the manufacture of a medicament for the treatment of an autoimmune disease or disorder.

One aspect of the present invention pertains to a crystalline salt, or pharmaceutical composition, each as described herein, in the manufacture of a medicament for the treatment of an inflammatory disease or disorder.

One aspect of the present invention pertains to a crystalline salt, or pharmaceutical composition, each as described herein, in the manufacture of a medicament for the treatment of a microbial infection or disease.

One aspect of the present invention pertains to a crystalline salt, or pharmaceutical composition, each as described herein, in the manufacture of a medicament for the treatment of a viral infection or disease.

One aspect of the present invention pertains to a crystalline salt, or pharmaceutical composition, each as described herein, in the manufacture of a medicament for the treatment of cancer.

One aspect of the present invention pertains to a crystalline salt, or pharmaceutical composition, each as described herein, in the manufacture of a medicament for the treatment of an S1P1 receptor-associated disorder selected from the group consisting of psoriasis, rheumatoid arthritis, Crohn's disease, transplant rejection, multiple sclerosis, systemic lupus erythematosus, ulcerative colitis, type I diabetes, acne, myocardial ischemia-reperfusion injury, hypertensive nephropathy, glomerulosclerosis, gastritis, polymyositis, thyroiditis, vitiligo, hepatitis, and biliary cirrhosis.

One aspect of the present invention pertains to a crystalline salt, or pharmaceutical composition, each as described herein, for use in a method for the treatment of the human or animal body by therapy.

One aspect of the present invention pertains to a crystalline salt, or pharmaceutical composition, each as described herein, for use in a method for the treatment of an S1P1 receptor-associated disorder.

One aspect of the present invention pertains to a crystalline salt, or pharmaceutical composition, each as described herein, for use in a method for the treatment of a disease or disorder mediated by lymphocytes.

One aspect of the present invention pertains to a crystalline salt, or pharmaceutical composition, each as described herein, for use in a method for the treatment of an autoimmune disease or disorder.

One aspect of the present invention pertains to a crystalline salt, or pharmaceutical composition, each as described herein, for use in a method for the treatment of an inflammatory disease or disorder.

One aspect of the present invention pertains to a crystalline salt, or pharmaceutical composition, each as described herein, for use in a method for the treatment of a microbial infection or disease.

One aspect of the present invention pertains to a crystalline salt, or pharmaceutical composition, each as described herein, for use in a method for the treatment of a viral infection or disease.

One aspect of the present invention pertains to a crystalline salt, or pharmaceutical composition, each as described herein, for use in a method for the treatment of cancer.

One aspect of the present invention pertains to a crystalline salt, or pharmaceutical composition, each as described herein, for use in a method for the treatment of an S1P1 receptor-associated disorder selected from the group consisting of psoriasis, rheumatoid arthritis, Crohn's disease, transplant rejection, multiple sclerosis, systemic lupus erythematosus, ulcerative colitis, type I diabetes, acne, myocardial ischemia-reperfusion injury, hypertensive nephropathy, glomerulosclerosis, gastritis, polymyositis, thyroiditis, vitiligo, hepatitis, and biliary cirrhosis.

One aspect of the present invention pertains to processes for preparing compositions comprising admixing a crystalline salt, each as described herein, and a pharmaceutically acceptable carrier.

One aspect of the present invention pertains to crystalline salts and pharmaceutical compositions of the present invention, for use in a method of treatment of the human or animal body by therapy.

### BRIEF DESCRIPTION OF THE DRAWINGS

FIG. 1: shows a powder X-ray diffraction (PXRD) pattern for a sample containing a crystalline Compound 1 diethanolamine Salt.

FIG. 2: shows a differential scanning calorimetry (DSC) thermogram for a sample containing crystalline Compound 1 diethanolamine Salt.

FIG. 3: shows a thermogravimetric analysis (TGA) thermogram of a sample containing anhydrous crystalline Compound 1 diethanolamine Salt.

FIG. 4: shows a powder X-ray diffraction (PXRD) pattern for a sample containing a crystalline Compound 1 diethylamine Salt.

FIG. 5: shows a differential scanning calorimetry (DSC) thermogram for a sample containing crystalline Compound 1 diethylamine Salt.

FIG. 6: shows a thermogravimetric analysis (TGA) thermogram of a sample containing anhydrous crystalline Compound 1 diethylamine Salt

### DETAILED DESCRIPTION

It should be appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

### Definitions

For clarity and consistency, the following definitions will be used throughout this patent document.

The term “agonist” refers to a moiety that interacts and activates the receptor, such as, the S1P1 receptor and initiate a physiological or pharmacological response characteristic of that receptor. For example, when moieties activate the intracellular response upon binding to the receptor, or enhance GTP binding to membranes.

The term “hydrate” as used refers to a compound of the invention or a salt thereof, that further includes a stoichiometric or non-stoichiometric amount of water bound by non-covalent intermolecular forces.

The term “in need of treatment” and the term “in need thereof,” when referring to treatment are used interchangeably to mean a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

The term “individual” refers to any animal, including mammals, preferably mice, rats, other rodents, rabbits, dogs, cats, swine, cattle, sheep, horses, or primates and most preferably humans.

The term “modulate or modulating” refers to an increase or decrease in the amount, quality, response or effect of a particular activity, function or molecule.

The term “pharmaceutical composition” refers to a composition comprising at least one active ingredient; including but not limited to, salts, solvates and hydrates of compounds of the present invention; whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “solvate” as used herein means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “therapeutically effective amount” refers to the amount of active compound or pharmaceutical agent that elicits the biological or medicinal response in a tissue, system, animal, individual or human that is being sought by a researcher, veterinarian, medical doctor or other clinician or caregiver; or in an individual, which includes one or more of the following:
(1) Preventing the disease; for example, preventing a disease, condition or disorder in an individual that may be predisposed to the disease, condition or disorder but does not yet experience or display the pathology or symptomatology of the disease,
(2) Inhibiting the disease; for example, inhibiting a disease, condition or disorder in an individual that is experiencing or displaying the pathology or symptomatology of the disease, condition or disorder (i.e., arresting further development of the pathology and/or symptomatology) and
(3) Ameliorating the disease; for example, ameliorating a disease, condition or disorder in an individual that is experiencing or displaying the pathology or symptomatology of the disease, condition or disorder (i.e., reversing the pathology and/or symptomatology).

The term “reacting” is used herein as known in the art and generally refers to the bringing together of chemical reagents in such a manner so as to allow their interaction at the molecular level to achieve a chemical or physical transformation of at least one chemical reagent.

### Processes

The present disclosure is directed to, inter alia, processes useful in the preparation of a crystalline form of (R)-2-(7-(4-cyclopentyl-3-(trifluoromethyl)benzyloxy)-1,2,3,4-tetrahydrocyclo-penta[b]indol-3-yl)acetic acid, for the treatment of an S1P1 receptor-associated disorder.

One aspect of the present disclosure relates to processes for preparing a crystalline salt of (R)-2-(7-(4-cyclopentyl-3-(trifluoromethyl)benzyloxy)-1,2,3,4-tetrahydrocyclo-penta[b]indol-3-yl)acetic acid comprising the steps of:
a) mixing (R)-2-(7-(4-cyclopentyl-3-(trifluoromethyl)benzyloxy)-1,2,3,4-tetrahydrocyclo-penta[b]indol-3-yl)acetic acid in an organic solvent resulting in a suspension;
b) adding an amine followed by water to the said suspension; and
c) isolating the crystalline salt.

One aspect of the present disclosure relates to processes for preparing a crystalline salt of (R)-2-(7-(4-cyclopentyl-3-(trifluoromethyl)benzyloxy)-1,2,3,4-tetrahydrocyclo-penta[b]indol-3-yl)acetic acid, wherein the organic solvent is selected from a group consisting of tetrahydrofuran (THF), 2-propanol, acetonitrile, ethyl acetate, ethanol, tert-butyl methyl ether (TBME), methyl isobutyl ketone (MIBK), water, and dimethyl sulfoxide (DMSO) and combinations thereof.

One aspect of the present disclosure relates to processes for preparing a crystalline salt of (R)-2-(7-(4-cyclopentyl-3-(trifluoromethyl)benzyloxy)-1,2,3,4-tetrahydrocyclo-penta[b]indol-3-yl)acetic acid, wherein the organic solvent is acetonitrile.

One aspect of the present disclosure relates to processes for preparing a crystalline salt of (R)-2-(7-(4-cyclopentyl-3-(trifluoromethyl)benzyloxy)-1,2,3,4-tetrahydrocyclo-penta[b]indol-3-yl)acetic acid, wherein the amine is selected from a group consisting of diethylamine, dimethylethanolamine, ethanolamine, diethanolamine, morpholine, tromethamine, N-methyl-D-glucamine, diethylethanolamine, and pyrrolidine.

One aspect of the present disclosure relates to processes for preparing a crystalline salt of (R)-2-(7-(4-cyclopentyl-3-(trifluoromethyl)benzyloxy)-1,2,3,4-tetrahydrocyclo-penta[b]indol-3-yl)acetic acid, wherein the amine is diethylamine.

One aspect of the present disclosure relates to processes for preparing a crystalline salt of (R)-2-(7-(4-cyclopentyl-3-(trifluoromethyl)benzyloxy)-1,2,3,4-tetrahydrocyclo-penta[b]indol-3-yl)acetic acid, wherein the amine is diethanolamine.

One aspect of the present disclosure relates to processes for preparing a crystalline salt of (R)-2-(7-(4-cyclopentyl-3-(trifluoromethyl)benzyloxy)-1,2,3,4 tetrahydrocyclo-penta[b]indol-3-yl)acetic acid, wherein said isolating step further comprises the steps of:
a) adding crystalline seed of (R)-2-(7-(4-cyclopentyl-3-(trifluoromethyl)benzyloxy)-1,2,3,4-tetrahydrocyclo-penta[b]indol-3-yl)acetic acid followed by stirring; and
b) cycling the temperature between 5° C. and 40° C. with a heat/cool rate of 0.1° C./minute and one hour hold at each temperature with stirring for 24 hours; and
c) collecting the precipitated solid by filtration.

One aspect of the present disclosure relates to processes for preparing a crystalline salt of (R)-2-(7-(4-cyclopentyl-3-(trifluoromethyl)benzyloxy)-1,2,3,4-tetrahydrocyclo-penta[b]indol-3-yl)acetic acid prepared by a process described herein.

One aspect of the present disclosure relates to processes of making a composition comprising mixing a crystalline form of (R)-2-(7-(4-cyclopentyl-3-(trifluoromethyl)benzyloxy)-1,2,3,4-tetrahydrocyclo-penta[b]indol-3-yl)acetic acid as described herein with a phamaceutically acceptable carrier.

One aspect of the present disclosure relates to processes of making a composition further comprising forming the composition into drug product, such as, a tablet, a pill, a powder, a lozenge, a sachet, a cachet, an elixir, a suspension, an emulsion, a solution, a syrup, a soft gelatin capsule, a hard gelatin capsule, a suppository, a sterile injectable solution, or a sterile packaged powder.

### Salts of the Present Invention

The present invention is directed, inter alia, to solid, stable, and readily isolable salts of (R)-2-(7-(4-cyclopentyl-3-(trifluoromethyl)benzyloxy)-1,2,3,4-tetrahydrocyclo-penta[b]indol-3-yl)acetic acid (Compound 1) and pharmaceutically acceptable solvates and hydrates thereof. The solid-state properties of the crystalline forms of salts the present invention is summarized infra.

One aspect of the present invention pertains to salts selected from: diethanolamine salt; diethylamine salt; and pharmaceutically acceptable solvates and hydrates thereof.

One aspect of the present invention pertains to diethanolamine salt.

One aspect of the present invention pertains to diethylamine salt.

One aspect of the present invention pertains to pharmaceutical compositions comprising a salt of the present invention.

One aspect of the present invention pertains to process for preparing a pharmaceutical composition comprising admixing a salt of the present invention and a pharmaceutically acceptable carrier.

### Crystalline Salts

Polymorphism is the ability of a substance to exist as two or more crystalline phases that have different arrangements and/or conformations of the molecules in the crystal lattice. Polymorphs show the same properties in the liquid or gaseous state but they may behave differently in the solid state.

Besides single-component polymorphs, drugs can also exist as salts and other multicomponent crystalline phases. For example, solvates and hydrates may contain an API host and either solvent or water molecules, respectively, as guests. Analogously, when the guest compound is a solid at room temperature, the resulting form is often called a cocrystal. Salts, solvates, hydrates, and cocrystals may show polymorphism as well. Crystalline phases that share the same API host, but differ with respect to their guests, may be referred to as pseudopolymorphs of one another.

Solvates contain molecules of the solvent of crystallization in a definite crystal lattice. Solvates, in which the solvent of crystallization is water, are termed hydrates. Because water is a constituent of the atmosphere, hydrates of drugs may be formed rather easily. Recently, polymorph screens of 245 compounds revealed that about 90% of them exhibited multiple solid forms. Overall, approximately half the compounds were polymorphic, often having one to three forms. About one-third of the compounds formed hydrates, and about one-third formed solvates. Data from cocrystal screens of 64 compounds showed that 60% formed cocrystals other than hydrates or solvates. (G. P. Stahly, Crystal Growth & Design (2007), 7(6), 1007-1026.)

The present invention is directed, inter alia, to crystalline salts of (R)-2-(7-(4-cyclopentyl-3-(trifluoromethyl)benzyloxy)-1,2,3,4-tetrahydrocyclo-penta[b]indol-3-yl)acetic acid (Compound 1) and hydrates and solvates thereof. The crystalline forms of the salts of the present invention can be identified by unique solid-state signatures with respect to, for example, differential scanning calorimetry (DSC), X-ray powder diffraction (PXRD), and other solid state methods. Further characterization with respect to water or solvent content of the crystalline salts of the present invention can be gauged by any of the following methods for example, thermogravimetric analysis (TGA), DSC and the like. For DSC, it is known that the temperatures observed will depend upon sample purity, the rate of temperature change, as well as sample preparation technique and the particular instrument employed. Thus, the values reported herein relating to DSC thermograms can vary by about ±6° C. The values reported herein relating to DSC thermograms can also vary by about ±20 joules per gram. For PXRD, the relative intensities of the peaks can vary, depending upon the sample preparation technique, the sample mounting procedure and the particular instrument employed. Moreover, instrument variation and other factors can often affect the 2θvalues. Therefore, the peak assignments of diffraction patterns can vary by about ±0.2°±2θ. The relative intensities of the reported peaks can also vary. For TGA, the features reported herein can vary by about ±5° C. The TGA features reported herein can also vary by about ±2% weight change due to, for example, sample variation. Further characterization with respect to hygroscopicity of the crystalline salt can be gauged by, for example, dynamic moisture sorption (DMS). The DMS features reported herein can vary by about ±5% relative humidity. The DMS features reported herein can also vary by about ±5% weight change. The deliquescence relative humidity (DRH) measurements by water activity meter are sensitive to sample quality and quantity. The DRH measurements reported herein can vary by about ±5% RH.

### Compound 1 Diethanolamine Salt.

One aspect of the present invention pertains to (R)-2-(7-(4-cyclopentyl-3-(trifluoromethyl)benzyloxy)-1,2,3,4-tetrahydrocyclo-penta[b]indol-3-yl)acetic acid diethanolamine salt, (Compound 1 Diethanolamine salt). The physical properties of Compound 1 Diethanolamine salt, are summarized in Table 1 below.

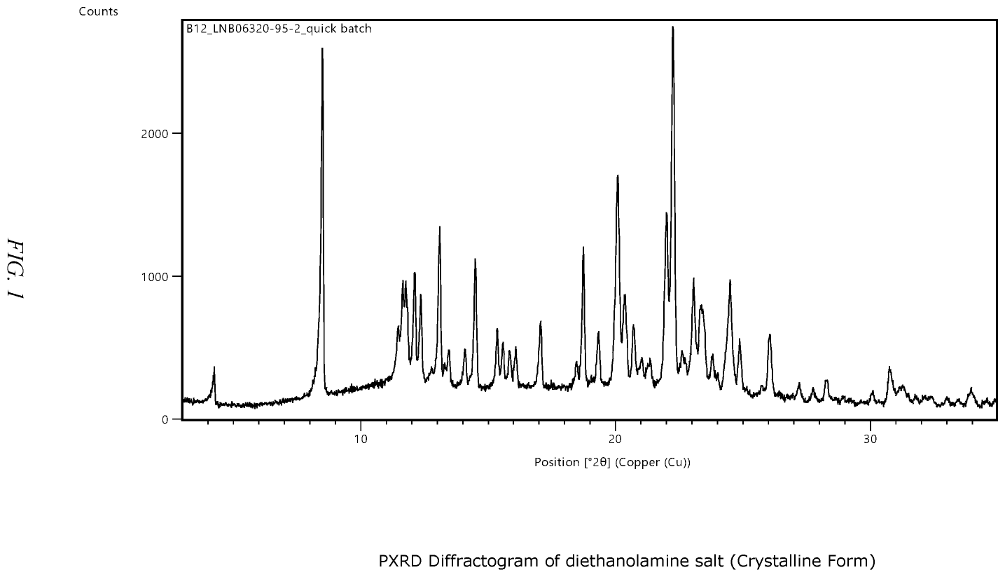

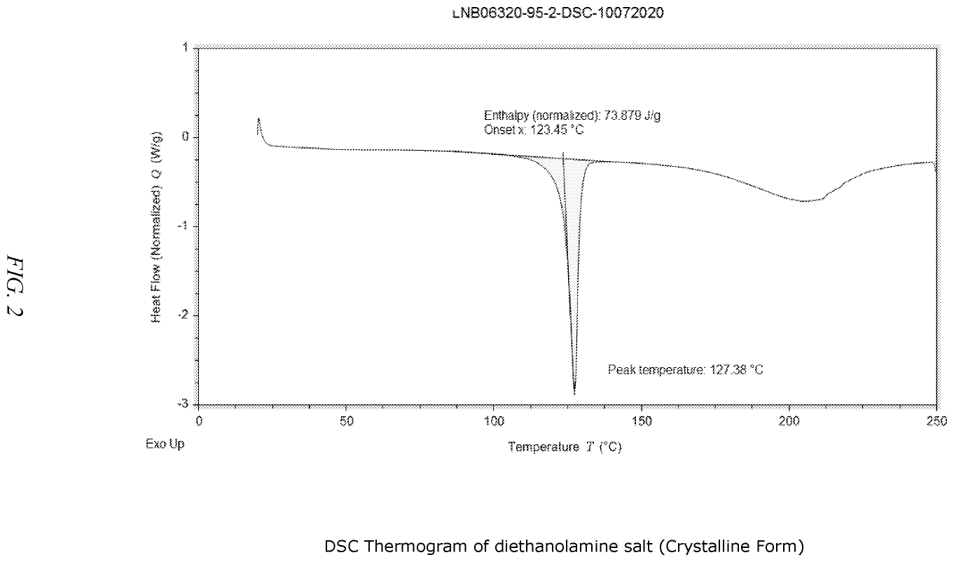

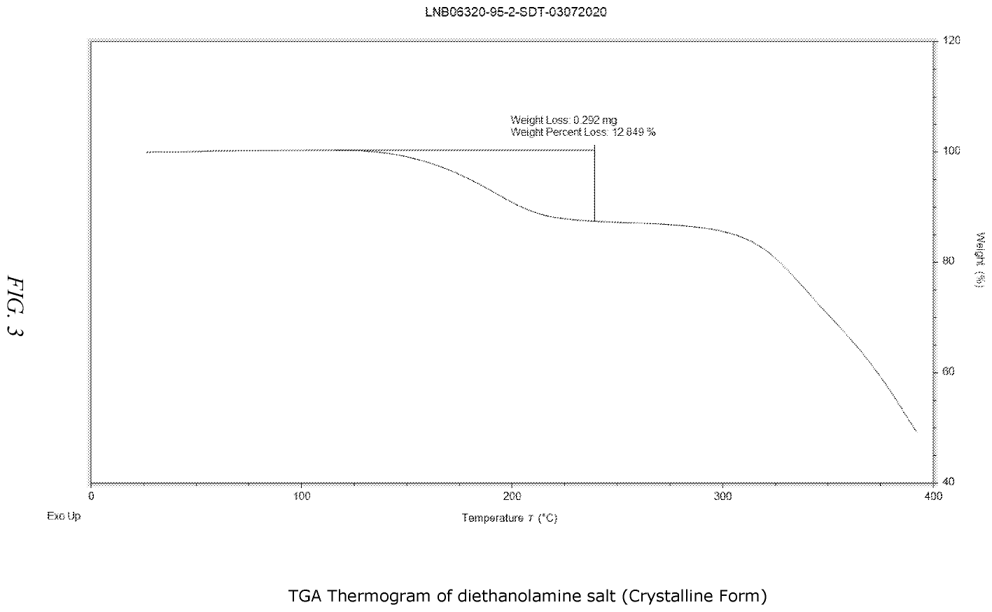

TABLE 1

| | Compound 1 Diethanolamine Salt |
|---|---|
| PXRD | FIG. 1: Peaks of >10% relative intensity at 8.5° ± |
| | 0.2°, 11.6° ± 0.2°, 11.8° ± 0.2°, 12.1° ± 0.2°, 13.1° ± |
| | 0.2°, 14.5° ± 0.2°, 18.7° ± 0.2°, 20.1° ± 0.2°, |
| | 20.4° ± 0.2°, 22.0° ± 0.2°, 22.2° ± 0.2°, 23.1° ± |
| | 0.2°, and 24.5° ± 0.2° θ |
| TGA | FIG. 3: Decrease in weight of about 12.8% out to |
| | about 130° C. |
| DSC | FIG. 2: Endotherms extrapolated onset |
| | temperature at about 123.5° C. |

Compound 1 diethanolamine salt showed weight loss by TGA (12.8%) accounting for loss of 0.8 equivalent of diethanolamine. The melting onset by DSC was 123.5° C.

Certain X-ray powder diffraction peaks for Compound 1 diethanolamine salt are shown in Table 2 below.

TABLE 2

| Pos. [°2 [symbol] .] | d-spacing [Å] | Rel. Int. [%] |
|---|---|---|
| 4.25 | 20.81 | 9.79 |
| 8.51 | 10.39 | 92.87 |
| 11.64 | 7.60 | 27.96 |
| 11.77 | 7.52 | 29.28 |
| 12.13 | 7.30 | 31.31 |
| 12.36 | 7.16 | 25.78 |
| 13.10 | 6.76 | 42.71 |
| 13.48 | 6.57 | 10.74 |
| 14.10 | 6.28 | 10.99 |
| 14.50 | 6.11 | 35.67 |
| 15.37 | 5.77 | 16.89 |
| 15.59 | 5.68 | 12.42 |
| 15.82 | 5.60 | 10.99 |
| 16.09 | 5.51 | 10.99 |
| 17.07 | 5.19 | 18.49 |
| 18.47 | 4.80 | 8.62 |
| 18.73 | 4.74 | 39.74 |
| 19.33 | 4.59 | 17.15 |
| 20.09 | 4.42 | 59.82 |
| 20.38 | 4.36 | 27.10 |
| 20.70 | 4.29 | 19.18 |
| 21.03 | 4.22 | 10.01 |
| 21.38 | 4.16 | 9.39 |
| 22.01 | 4.04 | 49.50 |
| 22.24 | 4.00 | 100.00 |
| 22.61 | 3.93 | 12.32 |
| 23.06 | 3.86 | 32.46 |
| 23.33 | 3.81 | 24.18 |
| 23.47 | 3.79 | 20.34 |
| 23.82 | 3.73 | 11.82 |
| 24.50 | 3.63 | 32.13 |
| 24.87 | 3.58 | 16.24 |
| 26.0 | 3.43 | 15.94 |
| 27.2 | 3.28 | 4.30 |
| 27.74 | 3.22 | 2.93 |
| 28.29 | 3.16 | 5.69 |
| 30.08 | 2.97 | 2.54 |
| 30.74 | 2.91 | 9.11 |
| 31.25 | 2.86 | 4.21 |
| 32.4 | 2.76 | 1.58 |
| 33.0 | 2.71 | 1.38 |
| 33.46 | 2.68 | 0.80 |
| 33.94 | 2.64 | 3.45 |

One aspect of the present disclosure relates to a diethanolamine salt having an X-ray powder diffraction pattern comprising a peak, in terms of 2θ, at 8.5°±0.2°, and 11.6°±0.2°. In some embodiments, the diethanolamine salt has an X-ray powder diffraction pattern comprising peaks, in terms of 2θ, at 8.5°±0.2°, 11.6°±0.2°, 11.8°±0.2°, and 12.1°±0.2°. In some embodiments, the diethanolamine salt has an X-ray powder diffraction pattern comprising peaks, in terms of 2θ, at 8.5°±0.2°, 11.6°±±0.2°, 11.8°±0.2°, 12.1°±0.2°, 13.1±0.2°, 14.5°±0.2°, and 18.7°±0.2°. In some embodiments, the diethanolamine salt has an X-ray powder diffraction pattern comprising peaks, in terms of 2θ, at 8.5° 0.2°, 11.6°±0.2°, 11.8°±0.2°, 12.1°±0.2°, 13.1°±0.2°, 14.5°±0.2°, 18.7°±0.2°, 20.1°±0.2°, 20.4°±0.2°, and 22.0°±0.2°. In some embodiments, the diethanolamine salt has an X-ray powder diffraction pattern comprising peaks, in terms of 2θ, at 8.5°±0.2°, 11.6°±0.2°, 11.8° 0.2°, 12.1°±0.2°, 13.1° 0.2°, 14.5±0.2°, 18.7°±0.2°, 20.1° 0.2°, 20.4°±0.2°, 22.0°±0.2°, 22.2°±0.2°, 23.1°±0.2°, and 24.5°±0.2°. In some embodiments, the diethanolamine salt has an X-ray powder diffraction pattern substantially as shown in FIG. 1, wherein by “substantially” is meant that the reported peaks can vary by about ±0.2° 20.

In some embodiments, the diethanolamine salt has a differential scanning calorimetry thermogram comprising an endotherm with an extrapolated onset temperature between about 120.5° C. and about 130.4° C. In some embodiments, the diethanolamine salt has a differential scanning calorimetry thermogram comprising an endotherm with an extrapolated onset temperature between about 120.5° C. and about 129.4° C. In some embodiments, the diethanolamine salt has a differential scanning calorimetry thermogram comprising an endotherm with an extrapolated onset temperature between about 121.5° C. and about 129.4° C. In some embodiments, the diethanolamine salt has a differential scanning calorimetry thermogram comprising an endotherm with an extrapolated onset temperature between about 121.5° C. and about 128.4° C. In some embodiments, the diethanolamine salt has a differential scanning calorimetry thermogram comprising an endotherm with an extrapolated onset temperature between about 122.5° C. and about 128.4° C. In some embodiments, the diethanolamine salt has a differential scanning calorimetry thermogram comprising an endotherm with an extrapolated onset temperature at about 123.5° C. In some embodiments, the diethanolamine salt has a differential scanning calorimetry thermogram substantially as shown in FIG. 2, wherein by “substantially” is meant that the reported DSC features can vary by about ±4° C. and that the reported DSC features can vary by about ±20 joules per gram.

In some embodiments, the diethanolamine salt has a thermogravimetric analysis profile showing about 14.8% weight loss below about 130° C. In some embodiments, the diethanolamine salt has a thermogravimetric analysis profile showing about 13.8% weight loss below about 130° C. In some embodiments, the diethanolamine salt has a thermogravimetric analysis profile showing about 12.8% weight loss below about 130° C. In some embodiments, the diethanolamine salt has a thermogravimetric analysis profile substantially as shown in FIG. 3, wherein by “substantially” is meant that the reported TGA features can vary by about ±5° C., and that that the reported TGA features can vary by about ±2% weight change.

One aspect of the present disclosure relates to the diethanolamine salt having:
a) an X-ray powder diffraction pattern comprising peaks, in terms of 2θ, at 8.5°±0.2°, and 11.6°±0.2°:
b) a differential scanning calorimetry thermogram comprising an endotherm with an extrapolated onset temperature between about 120.5° C. and about 130.4° C.; and/or

One aspect of the present disclosure relates to the diethanolamine salt having:
a) an X-ray powder diffraction pattern comprising peaks, in terms of 20, at 8.5±0.2°, 11.6°±0.2°, 11.8°±0.2°, and 12.1°±0.2°;
b) a differential scanning calorimetry thermogram comprising an endotherm with an extrapolated onset temperature between about 120.5° C. and about 129.4° C.; and/or

One aspect of the present disclosure relates to the diethanolamine salt having:
a) an X-ray powder diffraction pattern comprising peaks, in terms of 2θ, at 8.5±0.2°, 11.6°±0.2°, 11.8°±0.2°, 12.1°±0.2°, 13.1°±0.2°, 14.5±0.2°, and 18.7°±0.2°;
b) a differential scanning calorimetry thermogram comprising an endotherm with an extrapolated onset temperature between about 121.5° C. and about 129.4° C.; and/or

One aspect of the present disclosure relates to the diethanolamine salt having:
a) an X-ray powder diffraction pattern comprising peaks, in terms of 2θ, at 8.5°±0.2°, and 11.6°±0.2°;
b) a differential scanning calorimetry thermogram comprising an endotherm with an extrapolated onset temperature between about 120.5° C. and about 130.4° C.; and/or

One aspect of the present disclosure relates to the diethanolamine salt having:
a) an X-ray powder diffraction pattern comprising peaks, in terms of 2θ, at 8.5±0.2°, 11.6°±0.2°, 11.8°±0.2°, 12.1°±0.2°, 13.1°±0.2°, 14.5±0.2°, 18.7°±0.2°, 20.1°±0.2°, 20.4°±0.2°, and 22.0°±0.2°;
b) a differential scanning calorimetry thermogram comprising an endotherm with an extrapolated onset temperature between about 121.5° C. and about 128.4° C.; and/or
c) a thermogravimetric analysis profile showing about 14.8% weight loss below about 130° C.; and/or

One aspect of the present disclosure relates to the diethanolamine salt having:
a) an X-ray powder diffraction pattern comprising peaks, in terms of 2θ, at 8.5±0.2°, 11.6°±0.2°, 11.8°±0.2°, 12.1°±0.2°, 13.1°±0.2°, 14.5±0.2°, 18.7°±0.2°, 20.1°±0.2°, 20.4°±0.2°, 22.0°±0.2°, 22.2°±0.2°, 23.1°±0.2°, and 24.5°±0.2°;
b) a differential scanning calorimetry thermogram comprising an endotherm with an extrapolated onset temperature between about 122.5° C. and about 128.4° C.; and/or
c) a thermogravimetric analysis profile showing about 13.8% weight loss below about 130° C.; and/or

One aspect of the present disclosure relates to the diethanolamine salt having:
a) an X-ray powder diffraction pattern comprising peaks, in terms of 2θ, at 8.5°±0.2°, 11.6°±0.2°, 11.8°±0.2°, 12.1°±0.2°, 13.1°±0.2°, 14.5°±0.2°, 18.7°±0.2°, 20.1°±0.2°, 20.4°±0.2°, 22.0°±0.2°, 22.2°±0.2°, 23.1°±0.2°, and 24.5°±0.2°;
b) a differential scanning calorimetry thermogram comprising an endotherm with an extrapolated onset temperature at about 123.5° C.; and/or
c) a thermogravimetric analysis profile showing about 12.8% weight loss below about 130° C.; and/or

One aspect of the present disclosure relates to the diethanolamine salt having:
a) an X-ray powder diffraction pattern substantially as shown in FIG. 1;
b) a differential scanning calorimetry thermogram substantially as shown in FIG. 2; and/or
c) a thermogravimetric analysis profile substantially as shown in FIG. 3.

### Compound 1 Diethylamine Salt.

One aspect of the present invention pertains to (R)-2-(7-(4-cyclopentyl-3-(trifluoromethyl)benzyloxy)-1,2,3,4-tetrahydrocyclo-penta[b]indol-3-yl)acetic acid diethylamine salt, (Compound 1 diethylamine salt). The physical properties of Compound 1 Diethylamine salt, are summarized in Table 3 below.

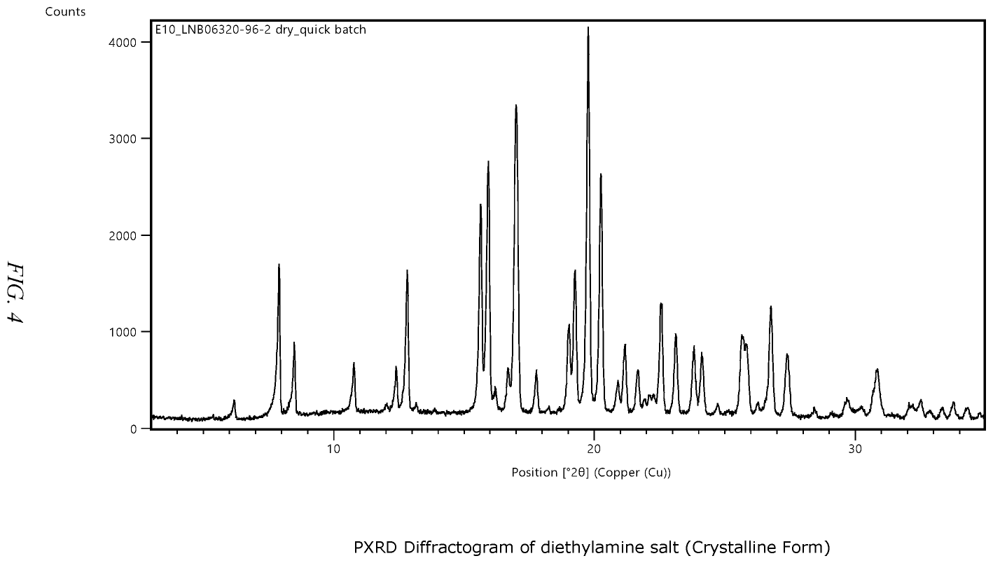

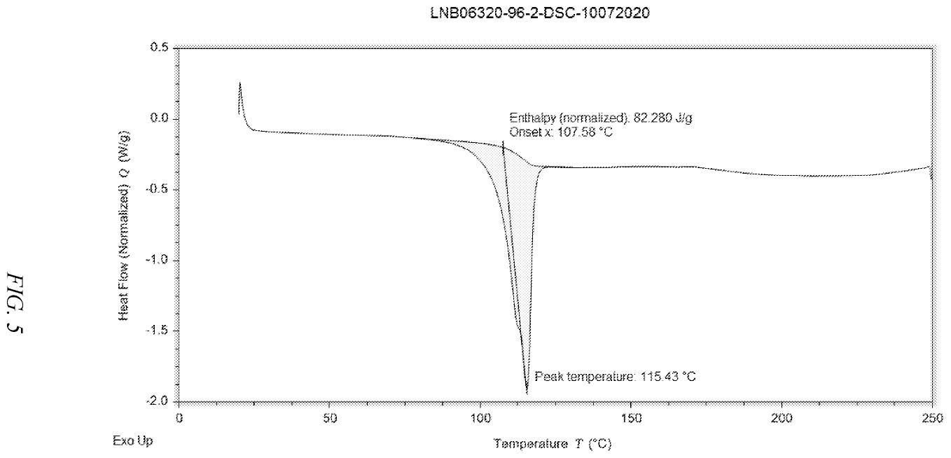

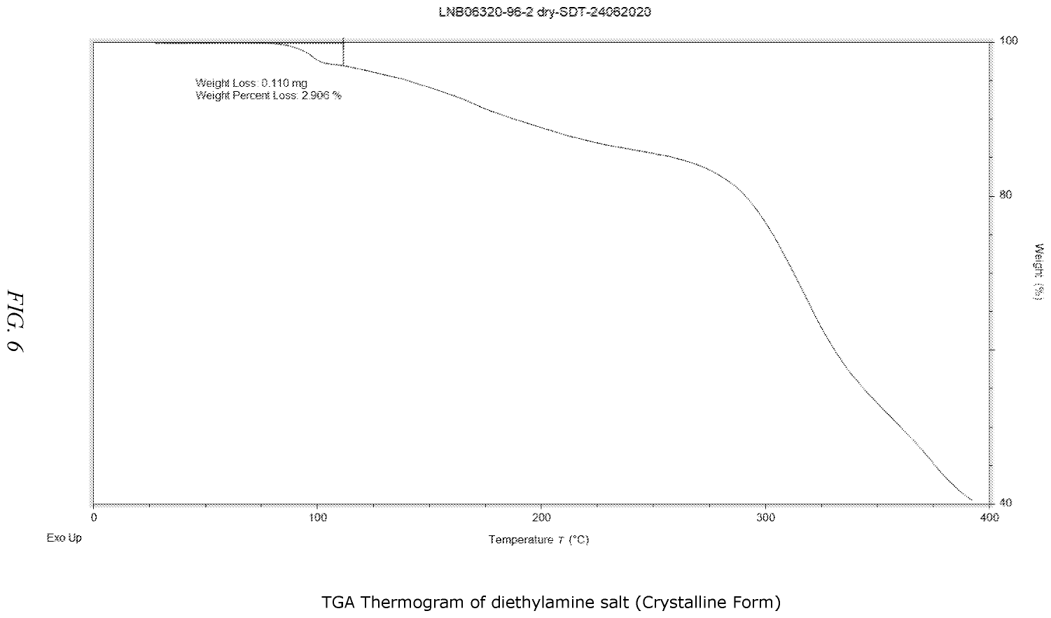

TABLE 3

| | Compound 1 Diethylamine Salt |
|---|---|
| PXRD | FIG. 4: Peaks of >10% relative intensity at 7.9° ± |
| | 0.2°, 8.5° ± 0.2°, 12.8° ± 0.2°, 15.6° ± 0.2°, 15.9° ± |
| | 0.2°, 17.0° ± 0.2°, 19.0° ± 0.2°, 19.3° ± 0.2°, 19.8° ± |
| | 0.2°, 20.3° ± 0.2°, 22.5° ± 0.2°, 23.1° ± 0.2°, and |
| | 26.8° ± 0.2° θ. |
| TGA | FIG. 6: Decrease in weight of about 2.9% out to |
| | about 110° C. |
| DSC | FIG. 5: Endotherm extrapolated onset temperature |
| | at about 107.6° C. |

Compound 1 diethylamine salt showed weight loss by TGA (2.9%) accounting for loss of 0.4 equivalent of acetonitrile or 1.3 equivalents water. The melting onset by DSC was 108° C.

Certain X-ray powder diffraction peaks for Compound 1 diethylamine salt are shown in Table 4 below.

TABLE 4

| Pos. [°2 [symbol] .] | d-spacing [Å] | Rel. Int. [%] |
|---|---|---|
| 6.19 | 14.28 | 4.85 |
| 7.91 | 11.17 | 38.73 |
| 8.49 | 10.41 | 19.79 |
| 10.78 | 8.21 | 13.73 |
| 12.41 | 7.14 | 12.39 |
| 12.83 | 6.90 | 37.82 |
| 13.17 | 6.72 | 3.80 |
| 15.64 | 5.67 | 55.86 |
| 15.95 | 5.56 | 63.93 |
| 16.21 | 5.47 | 7.84 |
| 16.69 | 5.31 | 13.05 |
| 16.99 | 5.22 | 80.82 |
| 17.78 | 4.99 | 11.97 |
| 18.22 | 4.87 | 2.95 |
| 19.03 | 4.66 | 24.20 |
| 19.27 | 4.61 | 37.97 |
| 19.77 | 4.49 | 100.00 |
| 20.26 | 4.38 | 62.65 |
| 20.91 | 4.25 | 9.96 |
| 21.18 | 4.19 | 19.12 |
| 21.68 | 4.10 | 12.73 |
| 21.92 | 4.05 | 5.14 |
| 22.11 | 4.02 | 6.20 |
| 22.53 | 3.95 | 29.54 |
| 23.13 | 3.85 | 21.64 |
| 23.82 | 3.74 | 18.94 |
| 24.11 | 3.69 | 17.24 |
| 24.73 | 3.60 | 3.86 |
| 25.63 | 3.48 | 19.59 |
| 25.86 | 3.45 | 18.19 |
| 26.26 | 3.39 | 4.14 |
| 26.77 | 3.33 | 29.11 |
| 27.40 | 3.26 | 16.34 |
| 28.45 | 3.14 | 2.42 |
| 29.10 | 3.07 | 1.44 |
| 29.72 | 3.01 | 4.90 |
| 30.25 | 2.95 | 3.15 |
| 30.86 | 2.90 | 12.89 |
| 32.10 | 2.79 | 3.21 |
| 32.52 | 2.75 | 4.93 |
| 32.86 | 2.73 | 2.05 |
| 33.33 | 2.69 | 2.85 |
| 33.77 | 2.65 | 4.38 |
| 34.32 | 2.61 | 2.98 |
| 34.77 | 2.58 | 1.58 |

One aspect of the present disclosure relates to a diethylamine salt having an X-ray powder diffraction pattern comprising a peak, in terms of 2θ, at 7.9°±0.2°, and 8.5°±0.2°. In some embodiments, the diethylamine salt has an X-ray powder diffraction pattern comprising peaks, in terms of 2θ, at 7.9°±0.2°, 8.5°±0.2°, 12.8°±0.2°, and 15.6°±0.2°.

In some embodiments, the diethylamine salt has an X-ray powder diffraction pattern comprising peaks, in terms of 2θ, at 7.9°±0.2°, 8.5°±0.2°, 12.8°±0.2°, 15.6°±0.2°, 15.90±0.2°, and 17.0°±0.2°.

In some embodiments, the diethylamine salt has an X-ray powder diffraction pattern comprising peaks, in terms of 2θ, at 7.9°±0.2°, 8.5°±0.2°, 12.8°±0.2°, 15.6°±0.2°, 15.9°±0.2°, 17.0°±0.2°, 19.0°±0.2°, 19.3°±0.2°, and 19.8°±0.2°.

In some embodiments, the diethylamine salt has an X-ray powder diffraction pattern comprising peaks, in terms of 2θ, at 7.9°±0.2°, 8.5°±0.2°, 12.8°±0.2°, 15.6°±0.2°, 15.9°±0.2°, 17.0°±0.2°, 19.0°±0.2°, 19.3°±0.2°, 19.8°±0.2°, 20.3°±0.2°, 22.5°±0.2°, 23.1°±0.2°, and 26.8°±0.2°. In some embodiments, the diethylamine salt has an X-ray powder diffraction pattern substantially as shown in FIG. 4, wherein by “substantially” is meant that the reported peaks can vary by about ±0.2° 2θ.

In some embodiments, the diethylamine salt has a differential scanning calorimetry thermogram comprising an endotherm with an extrapolated onset temperature between about 104.6° C. and about 119.4° C. In some embodiments, the diethylamine salt has a differential scanning calorimetry thermogram comprising an endotherm with an extrapolated onset temperature between about 104.6° C. and about 118.4° C. In some embodiments, the diethylamine salt has a differential scanning calorimetry thermogram comprising an endotherm with an extrapolated onset temperature between about 105.6° C. and about 118.4° C. In some embodiments, the diethylamine salt has a differential scanning calorimetry thermogram comprising an endotherm with an extrapolated onset temperature between about 105.6° C. and about 117.4° C. In some embodiments, the diethylamine salt has a differential scanning calorimetry thermogram comprising an endotherm with an extrapolated onset temperature between about 106.6° C. and about 116.4° C. In some embodiments, the diethylamine salt has a differential scanning calorimetry thermogram comprising an endotherm with an extrapolated onset temperature at about 107.6° C. In some embodiments, the diethylamine salt has a differential scanning calorimetry thermogram substantially as shown in FIG. 5, wherein by “substantially” is meant that the reported DSC features can vary by about ±4° C. and that the reported DSC features can vary by about ±20 joules per gram.

In some embodiments, the diethylamine salt has a thermogravimetric analysis profile showing about 4.9% weight loss below about 110° C. In some embodiments, the diethylamine salt has a thermogravimetric analysis profile showing about 3.9% weight loss below about 110° C. In some embodiments, the diethylamine salt has a thermogravimetric analysis profile showing about 2.9% weight loss below about 110° C. In some embodiments, the diethylamine salt has a thermogravimetric analysis profile substantially as shown in FIG. 6, wherein by “substantially” is meant that the reported TGA features can vary by about ±5° C., and that that the reported TGA features can vary by about ±2% weight change.

One aspect of the present disclosure relates to the diethylamine salt having:
a) an X-ray powder diffraction pattern comprising peaks, in terms of 2θ, at 7.9°±0.2°, and 8.5°±0.2°;
b) a differential scanning calorimetry thermogram comprising an endotherm with an extrapolated onset temperature between about 104.6° C. and about 119.4° C.; and/or

One aspect of the present disclosure relates to the diethylamine salt having:
a) an X-ray powder diffraction pattern comprising peaks, in terms of 2θ, at 7.9°±0.2°, 8.5°±0.2°, 12.8°±0.2°, and 15.6°±0.2°;
b) a differential scanning calorimetry thermogram comprising an endotherm with an extrapolated onset temperature between about 104.6° C. and about 118.4° C.; and/or

One aspect of the present disclosure relates to the diethylamine salt having:
a) an X-ray powder diffraction pattern comprising peaks, in terms of 2θ, at 7.9°±0.2°, 8.5°±0.2°, 12.8°±0.2°, 15.6°±0.2°, 15.9°±0.2°, and 17.0°±0.2°;
b) a differential scanning calorimetry thermogram comprising an endotherm with an extrapolated onset temperature between about 105.6° C. and about 118.4° C.; and/or

One aspect of the present disclosure relates to the diethylamine salt having:
a) an X-ray powder diffraction pattern comprising peaks, in terms of 2θ, at 7.9°±0.2°, 8.5°±0.2°, 12.8°±0.2°, 15.6°±0.2°, 15.9°±0.2°, 17.0°±0.2°, 19.0°±0.2°, 19.3°±0.2°, and 19.8°±0.2°;
b) a differential scanning calorimetry thermogram comprising an endotherm with an extrapolated onset temperature between about 105.6° C. and about 117.4° C.; and/or
c) a thermogravimetric analysis profile showing about 4.9% weight loss below about 110° C.; and/or

One aspect of the present disclosure relates to the diethylamine salt having:
a) an X-ray powder diffraction pattern comprising peaks, in terms of 2θ, at 7.9°±0.2°, 8.5°±0.2°, 12.8°±0.2°, 15.6°±0.2°, 15.9°±0.2°, 17.0°±0.2°, 19.0°±0.2°, 19.3°±0.2°, 19.8°±0.2°, 20.3°±0.2°, 22.5°±0.2°, 23.1°±0.2°, and 26.8°±0.2°;
b) a differential scanning calorimetry thermogram comprising an endotherm with an extrapolated onset temperature between about 106.6° C. and about 116.4° C.; and/or
c) a thermogravimetric analysis profile showing about 3.9% weight loss below about 110° C.; and/or

One aspect of the present disclosure relates to the diethylamine salt having:
a) an X-ray powder diffraction pattern comprising peaks, in terms of 2θ, at 7.9°±0.2°, 8.5°±0.2°, 12.8°±0.2°, 15.6°±0.2°, 15.9°±0.2°, 17.0°±0.2°, 19.0°±0.2°, 19.3°±0.2°, 19.8°±0.2°, 20.3°±0.2°, 22.5°±0.2°, 23.1°±0.2°, and 26.8°±0.2°;
b) a differential scanning calorimetry thermogram comprising an endotherm with an extrapolated onset temperature at about 107.6° C.; and/or
c) a thermogravimetric analysis profile showing about 2.9% weight loss below about 110° C.; and/or

One aspect of the present disclosure relates to the diethylamine salt having:
a) an X-ray powder diffraction pattern substantially as shown in FIG. 4;
b) a differential scanning calorimetry thermogram substantially as shown in FIG. 5; and/or
c) a thermogravimetric analysis profile substantially as shown in FIG. 6.

One aspect of the present invention is directed to methods for treating an S1P1 receptor-associated disorder in an individual comprising administering to said individual in need thereof a therapeutically effective amount of a salt, a crystalline form, or a pharmaceutical composition as described herein.

One aspect of the present invention is directed to methods for treating psoriasis in an individual comprising administering to said individual in need thereof a therapeutically effective amount of a salt, a crystalline form, or a pharmaceutical composition as described herein.

One aspect of the present invention is directed to methods for treating rheumatoid arthritis in an individual comprising administering to said individual in need thereof a therapeutically effective amount of a salt, a crystalline form, or a pharmaceutical composition as described herein.

One aspect of the present invention is directed to methods for treating Crohn's disease in an individual comprising administering to said individual in need thereof a therapeutically effective amount of a salt, a crystalline form, or a pharmaceutical composition as described herein.

One aspect of the present invention is directed to methods for treating transplant rejection in an individual comprising administering to said individual in need thereof a therapeutically effective amount of a salt, a crystalline form, or a pharmaceutical composition as described herein.

One aspect of the present invention is directed to methods for treating multiple sclerosis in an individual comprising administering to said individual in need thereof a therapeutically effective amount of a salt, a crystalline form, or a pharmaceutical composition as described herein.

One aspect of the present invention is directed to methods for treating systemic lupus erythematosus in an individual comprising administering to said individual in need thereof a therapeutically effective amount of a salt, a crystalline form, or a pharmaceutical composition as described herein.

One aspect of the present invention is directed to methods for treating ulcerative colitis in an individual comprising administering to said individual in need thereof a therapeutically effective amount of a salt, a crystalline form, or a pharmaceutical composition as described herein.

One aspect of the present invention is directed to methods for treating type I diabetes in an individual comprising administering to said individual in need thereof a therapeutically effective amount of a salt, a crystalline form, or a pharmaceutical composition as described herein.

One aspect of the present invention is directed to methods for treating acne in an individual comprising administering to said individual in need thereof a therapeutically effective amount of a salt, a crystalline form, or a pharmaceutical composition as described herein.

One aspect of the present invention is directed to methods for treating myocardial ischemia-reperfusion injury in an individual comprising administering to said individual in need thereof a therapeutically effective amount of a salt, a crystalline form, or a pharmaceutical composition as described herein.

One aspect of the present invention is directed to methods for treating hypertensive nephropathy in an individual comprising administering to said individual in need thereof a therapeutically effective amount of a salt, a crystalline form, or a pharmaceutical composition as described herein.

One aspect of the present invention is directed to methods for treating glomerulosclerosis in an individual comprising administering to said individual in need thereof a therapeutically effective amount of a salt, a crystalline form, or a pharmaceutical composition as described herein.

One aspect of the present invention is directed to methods for treating gastritis in an individual comprising administering to said individual in need thereof a therapeutically effective amount of a salt, a crystalline form, or a pharmaceutical composition as described herein.

One aspect of the present invention is directed to methods for treating polymyositis in an individual comprising administering to said individual in need thereof a therapeutically effective amount of a salt, a crystalline form, or a pharmaceutical composition as described herein.

One aspect of the present invention is directed to methods for treating thyroiditis in an individual comprising administering to said individual in need thereof a therapeutically effective amount of a salt, a crystalline form, or a pharmaceutical composition as described herein.

One aspect of the present invention is directed to methods for treating vitiligo in an individual comprising administering to said individual in need thereof a therapeutically effective amount of a salt, a crystalline form, or a pharmaceutical composition as described herein.

One aspect of the present invention is directed to methods for treating hepatitis in an individual comprising administering to said individual in need thereof a therapeutically effective amount of a salt, a crystalline form, or a pharmaceutical composition as described herein.

One aspect of the present invention is directed to methods for treating biliary cirrhosis in an individual comprising administering to said individual in need thereof a therapeutically effective amount of a salt, a crystalline form, or a pharmaceutical composition as described herein.

One aspect of the present invention pertains to the use of a salt or a crystalline form, as described herein, in the manufacture of a medicament for the treatment of psoriasis.

One aspect of the present invention pertains to the use of a salt or a crystalline form, as described herein, in the manufacture of a medicament for the treatment of rheumatoid arthritis.

One aspect of the present invention pertains to the use of a salt or a crystalline form, as described herein, in the manufacture of a medicament for the treatment of Crohn's disease.

One aspect of the present invention pertains to the use of a salt or a crystalline form, as described herein, in the manufacture of a medicament for the treatment of transplant rejection.

One aspect of the present invention pertains to the use of a salt or a crystalline form, as described herein, in the manufacture of a medicament for the treatment of multiple sclerosis.

One aspect of the present invention pertains to the use of a salt or a crystalline form, as described herein, in the manufacture of a medicament for the treatment of systemic lupus erythematosus.

One aspect of the present invention pertains to the use of a salt or a crystalline form, as described herein, in the manufacture of a medicament for the treatment of ulcerative colitis.

One aspect of the present invention pertains to the use of a salt or a crystalline form, as described herein, in the manufacture of a medicament for the treatment of type I diabetes.

One aspect of the present invention pertains to the use of a salt or a crystalline form, as described herein, in the manufacture of a medicament for the treatment of acne.

One aspect of the present invention pertains to the use of a salt or a crystalline form, as described herein, in the manufacture of a medicament for the treatment of myocardial ischemia-reperfusion injury.

One aspect of the present invention pertains to the use of a salt or a crystalline form, as described herein, in the manufacture of a medicament for the treatment of hypertensive nephropathy.

One aspect of the present invention pertains to the use of a salt or a crystalline form, as described herein, in the manufacture of a medicament for the treatment of glomerulosclerosis.

One aspect of the present invention pertains to the use of a salt or a crystalline form, as described herein, in the manufacture of a medicament for the treatment of gastritis.

One aspect of the present invention pertains to the use of a salt or a crystalline form, as described herein, in the manufacture of a medicament for the treatment of polymyositis.

One aspect of the present invention pertains to the use of a salt or a crystalline form, as described herein, in the manufacture of a medicament for the treatment of thyroiditis.

One aspect of the present invention pertains to the use of a salt or a crystalline form, as described herein, in the manufacture of a medicament for the treatment of vitiligo.

One aspect of the present invention pertains to the use of a salt or a crystalline form, as described herein, in the manufacture of a medicament for the treatment of hepatitis.

One aspect of the present invention pertains to the use of a salt or a crystalline form, as described herein, in the manufacture of a medicament for the treatment of biliary cirrhosis.

One aspect of the present invention pertains to a salt, a crystalline form, or a pharmaceutical composition, as described herein, for use in a method for the treatment of the human or animal body by therapy.

One aspect of the present invention pertains to a salt, a crystalline form, or a pharmaceutical composition, as described herein, for use in a method for the treatment of psoriasis.

One aspect of the present invention pertains to a salt, a crystalline form, or a pharmaceutical composition, as described herein, for use in a method for the treatment of rheumatoid arthritis.

One aspect of the present invention pertains to a salt, a crystalline form, or a pharmaceutical composition, as described herein, for use in a method for the treatment of Crohn's disease.

One aspect of the present invention pertains to a salt, a crystalline form, or a pharmaceutical composition, as described herein, for use in a method for the treatment of transplant rejection.

One aspect of the present invention pertains to a salt, a crystalline form, or a pharmaceutical composition, as described herein, for use in a method for the treatment of multiple sclerosis.

One aspect of the present invention pertains to a salt, a crystalline form, or a pharmaceutical composition, as described herein, for use in a method for the treatment of systemic lupus erythematosus.

One aspect of the present invention pertains to a salt, a crystalline form, or a pharmaceutical composition, as described herein, for use in a method for the treatment of ulcerative colitis.

One aspect of the present invention pertains to a salt, a crystalline form, or a pharmaceutical composition, as described herein, for use in a method for the treatment of type I diabetes.

One aspect of the present invention pertains to a salt, a crystalline form, or a pharmaceutical composition, as described herein, for use in a method for the treatment of acne.

One aspect of the present invention pertains to a salt, a crystalline form, or a pharmaceutical composition, as described herein, for use in a method for the treatment of myocardial ischemia-reperfusion injury.

One aspect of the present invention pertains to a salt, a crystalline form, or a pharmaceutical composition, as described herein, for use in a method for the treatment of hypertensive nephropathy.

One aspect of the present invention pertains to a salt, a crystalline form, or a pharmaceutical composition, as described herein, for use in a method for the treatment of glomerulosclerosis.

One aspect of the present invention pertains to a salt, a crystalline form, or a pharmaceutical composition, as described herein, for use in a method for the treatment of gastritis.

One aspect of the present invention pertains to a salt, a crystalline form, or a pharmaceutical composition, as described herein, for use in a method for the treatment of polymyositis.

One aspect of the present invention pertains to a salt, a crystalline form, or a pharmaceutical composition, as described herein, for use in a method for the treatment of thyroiditis.

One aspect of the present invention pertains to a salt, a crystalline form, or a pharmaceutical composition, as described herein, for use in a method for the treatment of vitiligo.

One aspect of the present invention pertains to a salt, a crystalline form, or a pharmaceutical composition, as described herein, for use in a method for the treatment of hepatitis.

One aspect of the present invention pertains to a salt, a crystalline form, or a pharmaceutical composition, as described herein, for use in a method for the treatment of biliary cirrhosis.

### Pharmaceutical Compositions

A further aspect of the present invention pertains to pharmaceutical compositions comprising one or more salts according to any of the salt embodiments disclosed herein and one or more pharmaceutically acceptable carriers. Some embodiments pertain to pharmaceutical compositions comprising a salt according to any of the salt embodiments disclosed herein and a pharmaceutically acceptable carrier. Some embodiments pertain to pharmaceutical compositions comprising any sub-combination of salts according to any of the salt embodiments disclosed herein.

Another aspect of the present invention pertains to methods of producing pharmaceutical compositions comprising admixing one or more salts according to any of the salt embodiments disclosed herein and one or more pharmaceutically acceptable carriers. Some embodiments pertain to a method of producing a pharmaceutical composition comprising admixing a salt according to any of the salt embodiments disclosed herein and a pharmaceutically acceptable carrier. Some embodiments pertain to a methods of producing pharmaceutical compositions comprising admixing any sub-combination of salts according to any of the salt embodiments disclosed herein and a pharmaceutically acceptable carrier.

Formulations may be prepared by any suitable method, typically by uniformly mixing the active salt(s) with liquids or finely divided solid carriers, or both, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the salt of the invention in a suitable liquid vehicle and filter sterilizing the solution before filling and sealing an appropriate vial or ampule. These are just a few examples of the many appropriate methods well known in the art for preparing dosage forms.

The salts of the present invention can be formulated into pharmaceutical compositions and bulk pharmaceutical compositions suitable for the manufacture of dosage forms using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro et al.) While it is possible that, for use in the prophylaxis or treatment, a salt of the invention may, in an alternative use, be administered as a raw or pure chemical, it is preferable however to present the salt or active ingredient as a pharmaceutical formulation or composition further comprising a pharmaceutically acceptable carrier.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The salts of the invention, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention or a salt, solvate, hydrate or physiologically functional derivative thereof can be used as active ingredients in pharmaceutical compositions, specifically as S1P1 receptor modulators. By the term “active ingredient” is defined in the context of a “pharmaceutical composition” and refers to a component of a pharmaceutical composition that provides the primary pharmacological effect, as opposed to an “inactive ingredient” which would generally be recognized as providing no pharmaceutical benefit.

The dose when using the salts of the present invention can vary within wide limits and as is customary and is known to the physician, it is to be tailored to the individual conditions in each individual case. It depends, for example, on the nature and severity of the illness to be treated, on the condition of the patient, on the salt employed or on whether an acute or chronic disease state is treated or prophylaxis conducted or on whether further active compounds are administered in addition to the salts of the present invention. Representative doses of the present invention include, but are not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular salt employed, whether a drug delivery system is utilized, on whether an acute or chronic disease state is being treated or prophylaxis conducted or on whether further active compounds are administered in addition to the salts of the present invention and as part of a drug combination. The dosage regimen for treating a disease condition with the salts and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The salts of the present invention can be administrated in a wide variety of oral and parenteral dosage forms. It will be obvious to those skilled in the art that the following dosage forms may comprise, as the active component, either a salt of the invention or a solvate or hydrate of a salt of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets may contain varying percentage amounts of the active salt. A representative amount in a powder or tablet may contain from 0.5 to about 90 percent of the active salt; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” is intended to include the formulation of the active salt with encapsulating material as carrier providing a capsule in which the active component, with or without carriers, is surrounded by a carrier, which is thus in association with it. Similarly, cachets and lozenges are included. Tablets, powders, capsules, pills, cachets and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the salts according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the salts of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the salts of the present invention as an aerosol can be prepared by processes well known to the person skilled in the art. For their preparation, for example, solutions or dispersions of the salts of the present invention in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the salt will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the salt in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The present invention is directed to pharmaceutical compositions that include every combination of one or more of the salts, or crystalline forms selected from the following group:
(R)-2-(7-(4-cyclopentyl-3-(trifluoromethyl)benzyloxy)-1,2,3,4-tetrahydrocyclo-penta[b]indol-3-yl)acetic acid (Compound 1) diethanolamine salt;
(R)-2-(7-(4-cyclopentyl-3-(trifluoromethyl)benzyloxy)-1,2,3,4-tetrahydrocyclo-penta[b]indol-3-yl)acetic acid (Compound 1) diethylamine salt

One aspect of the present invention pertains to pharmaceutical compositions comprising (R)-2-(7-(4-cyclopentyl-3-(trifluoromethyl)benzyloxy)-1,2,3,4-tetrahydrocyclo-penta[b]indol-3-yl)acetic acid (Compound 1) diethanolamine salt and a pharmaceutically acceptable carrier.

One aspect of the present invention pertains to pharmaceutical compositions comprising a crystalline form of (R)-2-(7-(4-cyclopentyl-3-(trifluoromethyl)benzyloxy)-1,2,3,4-tetrahydrocyclo-penta[b]indol-3-yl)acetic acid (Compound 1) diethanolamine salt and a pharmaceutically acceptable carrier.

One aspect of the present invention pertains to pharmaceutical compositions comprising (R)-2-(7-(4-cyclopentyl-3-(trifluoromethyl)benzyloxy)-1,2,3,4-tetrahydrocyclo-penta[b]indol-3-yl)acetic acid (Compound 1) diethylamine salt and a pharmaceutically acceptable carrier.

One aspect of the present invention pertains to pharmaceutical compositions comprising a crystalline form of (R)-2-(7-(4-cyclopentyl-3-(trifluoromethyl)benzyloxy)-1,2,3,4-tetrahydrocyclo-penta[b]indol-3-yl)acetic acid (Compound 1) diethylamine salt and a pharmaceutically acceptable carrier.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987, both of which are hereby incorporated by reference in their entirety.

The embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier.

It is noted that when the S1P1 receptor modulators are utilized as active ingredients in a pharmaceutical composition, these are not intended for use only in humans, but in other non-human mammals as well. Indeed, recent advances in the area of animal health-care mandate that consideration be given for the use of active agents, such as S1P1 receptor modulators, for the treatment of an S1P-associated disease or disorder in companionship animals (e.g., cats, dogs, etc.) and in livestock animals (e.g., cows, chickens, fish, etc.) Those of ordinary skill in the art are readily credited with understanding the utility of such compounds in such settings.

### EXAMPLES

The following examples are provided to further define the invention without, however, limiting the invention to the particulars of these examples. The compounds and salts thereof described herein, supra and infra, are named according to the CS ChemDraw Ultra Version 7.0.1, AutoNom version 2.2, or CS ChemDraw Ultra Version 9.0.7. In certain instances common names are used and it is understood that these common names would be recognized by those skilled in the art.

Powder X-ray Diffraction (PXRD) analysis was carried out on a PANalytical X'pert pro with PIXcel detector (128 channels), scanning the samples between 3 and 35° 2θ. The material was gently ground to release any agglomerates and loaded onto a multi-well plate with Mylar polymer film to support the sample. The multi-well plate was then placed into the diffractometer and analysed using Cu K radiation (α₁ λ=1.54060 Å; α₂=1.54443 Å; β=1.39225 Å; α₁: α₂ ratio=0.5) running in transmission mode (step size 0.0130° 20, step time 18.87 s) using 40 kV/40 mA generator settings. Data were visualized and images generated using the HighScore Plus 4.8 desktop application (PANalytical, 2017)

Approximately, 1-5 mg of material was weighed into an aluminum DSC pan and sealed non-hermetically with an aluminum lid. The sample pan was then loaded into a TA Instruments Discovery DSC 2500 differential scanning calorimeter equipped with a RC90 cooler. The sample and reference were heated to 220° C. at a scan rate of 10° C./min and the resulting heat flow response monitored. The sample was re-cooled to 20° C. and then reheated again to 220° C. all at 10° C./min. Nitrogen was used as the purge gas, at a flow rate of 50 cm³/min.

Approximately, 5-10 mg of material was added into a pre-tared open aluminum pan and loaded into a TA Instruments Discovery SDT 650 Auto-Simultaneous DSC and held at room temperature. The sample was then heated at a rate of 10° C./min from 30° C. to 400° C. during which time the change in sample weight was recorded along with the heat flow response (DSC). Nitrogen was used as the sample purge gas, at a flow rate of 200 cm³/min.

### Example 1: Preparation of (R)-2-(7-(4-cyclopentyl-3-(trifluoromethyl)benzyloxy)-1,2,3,4-tetrahydrocyclo-penta[b]indol-3-yl)acetic acid (Compound 1) diethanolamine Salt (Compound 1 Diethanolamine Salt)

(R)-2-(7-(4-cyclopentyl-3-(trifluoromethyl)benzyloxy)-1,2,3,4-tetrahydrocyclo-penta[b]indol-3-yl)acetic acid (140 mg) was suspended in acetonitrile (400 μL) in a 20 mL vial.
Diethanolamine (1.05 eq.) was added followed by water (10 μL).
Crystalline seed (1-2 mg) was added followed by a stirrer bar was added and the vial sealed with parafilm.
A solid precipitated over a period of ca. 5 min and was isolated via centrifugation.

The isolated solid was dried under vacuum at ambient temperature for 24 hr, yielding an orange solid (165 mg, 96%).

The powder X-ray diffraction pattern of the title salt is shown in FIG. 1. Thermal analysis (DSC and TGA) of the title salt is shown in FIG. 2 and FIG. 3 respectively.

### Preparation of Crystalline Seed of Compound 1 Diethanolamine Salt

(R)-2-(7-(4-cyclopentyl-3-(trifluoromethyl)benzyloxy)-1,2,3,4-tetrahydrocyclo-penta[b]indol-3-yl)acetic acid (140 mg) was suspended in acetonitrile (400 μL) in a 20 mL vial.
Diethanolamine (1.05 eq.) was added followed by water (10 μL).
To the resulting mixture was added a stirrer bar and the vial sealed with parafilm.
The vial was sealed and temperature cycled between 5 and 40° C. with a heat/cool rate of 0.1° C./min and 1 hr holds at each temperature with stirring for 24 hr.
The mixture was cooled to 5° C.
The solid was isolated via centrifugation and then dried under vacuum at ambient temperature for 24 hr.

### Example 2: Preparation of (R)-2-(7-(4-cyclopentyl-3-(trifluoromethyl)benzyloxy)-1,2,3,4-tetrahydrocyclo-penta[b]indol-3-yl)acetic Acid (Compound 1) diethylamine Salt (Compound 1 Diethylamine Salt)

(R)-2-(7-(4-cyclopentyl-3-(trifluoromethyl)benzyloxy)-1,2,3,4-tetrahydrocyclo-penta[b]indol-3-yl)acetic acid (250 mg) was weighed into a 20 mL vial.
The sample was suspended in acetonitrile (1 mL).
Diethylamine 1.05 eq. was added.
Water (20 μL) was added to the diethylamine sample.
The vial was sealed and temperature cycled between 5 and 40° C. with a heat/cool rate of 0.1° C./min and 1 hr holds at each temperature with stirring for 24 hr.
Solid was isolated via centrifugation and dried under vacuum at ambient temperature for 24 hr.
The solid was further characterized by PXRD and TG/DSC.

The powder X-ray diffraction pattern of the title salt is shown in FIG. 4. Thermal analysis (DSC and TGA) of the title salt is shown in FIG. 5 and FIG. 6 respectively.

Those skilled in the art will recognize that various modifications, additions, substitutions, and variations to the illustrative examples set forth herein can be made without departing from the spirit of the invention and are, therefore, considered within the scope of the invention.

1. A crystalline salt selected from:
(R)-2-(7-(4-cyclopentyl-3-(trifluoromethyl)benzyloxy)-1,2,3,4-tetrahydrocyclo-penta[b]indol-3-yl)acetic acid diethanolamine salt; and
(R)-2-(7-(4-cyclopentyl-3-(trifluoromethyl)benzyloxy)-1,2,3,4-tetrahydrocyclo-penta[b]indol-3-yl)acetic acid diethylamine salt;
or pharmaceutically acceptable solvates and hydrates thereof,
wherein said diethanolamine salt has an X-ray powder diffraction pattern comprising peaks, in terms of 2θ6, at 8.5°±0.2°, 11.6°±0.2°, 11.8°±0.2°, and 12.10° 0.2°; and
wherein said diethylamine salt has an X-ray powder diffraction pattern comprising peaks, in terms of 2θ, at 7.9°±0.2°, 8.5°±0.2°, 12.8°±0.2°, and 15.6°±0.2°.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. A pharmaceutical composition comprising a crystalline salt according to claim 1, and a pharmaceutically acceptable carrier.

53. (canceled)

54. A method for treating an S1P1 receptor-associated disorder in an individual comprising administering to said individual in need thereof a therapeutically effective amount of a crystalline salt according to claim 1.

55. (canceled)

56. (canceled)

57. (canceled)

58. A method according claim 54, wherein said S1P1 receptor-associated disorder is selected from the group consisting of psoriasis, rheumatoid arthritis, Crohn's disease, transplant rejection, multiple sclerosis, systemic lupus erythematosus, ulcerative colitis, type I diabetes, acne, myocardial ischemia-reperfusion injury, hypertensive nephropathy, glomerulosclerosis, gastritis, polymyositis, thyroiditis, vitiligo, hepatitis, and biliary cirrhosis.

59. (canceled)

60. (canceled)

61. (canceled)

62. (canceled)

63. (canceled)

64. (canceled)

65. (canceled)
